(19) United States
(12) Patent Application Publication
(10) Pub. No.: US 2005/0085632 A1
(43) Pub. Date: Apr. 21, 2005

(54) Method of removing isoflavones and phytates

(76) Inventors: Paul W. Johns, Columbus, OH (US); Andre Daab-Krzykowski, Columbus, OH (US); Terrence B. Mazer, Reynoldsburg, OH (US); John D. Suh, Gahanna, OH (US)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 10/929,334

(22) Filed: Aug. 27, 2004

Related U.S. Application Data
(62) Division of application No. 09/946,876, filed on Sep. 5, 2001, now abandoned.

(51) Int. Cl. C07F 9/02; C07H 15/00; C07D 311/76

(52) U.S. Cl. 536/8; 549/403; 558/147

(57) ABSTRACT

Methods for sequentially removing isoflavones and phytates from an active surface by utilizing an aqueous medium for isoflavone removal and an aqueous medium for phytate removal. The aqueous medium for isoflavone removal contains at least one alcohol and at least one acid. The aqueous medium for phytate removal is either a relatively stronger acidic solution, a basic solution, or with some active surfaces may be an aqueous solution of pH 2-7, which is essentially free of alcohol and organic solvents. The use of the methods disclosed allows sequential isolation of isoflavones and phytates, compounds which may then be utilized in various foods for human consumption.

### CROSS REFERENCE

This application is a divisional of U.S. patent application Ser. No. 09/946,876, filed Sep. 5, 2001.

### FIELD OF THE INVENTION

The invention relates to methods for sequentially removing isoflavones and phytates from active surfaces.

### BACKGROUND

Plant proteins are frequently utilized as protein sources in food for human consumption such as nutritional formulas or cereals, but are often purified prior to such use. Purification may be utilized to remove compounds such as phytoestrogens or plant estrogens, manganese or nucleotides. Phytoestrogens are plant substances that are structurally and functionally similar to the gonadal steroid, 17 β-estradiol, that produce estrogenic effects. There are three main groups of nonsteroidal dietary estrogens: (1) isoflavones, (2) coumestans, and (3) mycoestrogens (fungal). A review of phytoestrogens and their effects in mammals is reported by Kaldas and Hughes in “Reproductive and General Metabolic Effects of Phytoestrogens in Mammals,” Reproductive Toxicology, vol. 3, pp. 81-89, 1989. As used herein, the term “isoflavones” is equivalent to the term “phytoestrogens” as the term is defined in the Kaldas et al. article. Thus, isoflavones include flavonones, flavonols, flavones, isoflavones, aurones, chalcones, dihydrochalcones, anthocyanins, leucoanthocyanins, leucoanthocyanidins, anthocyanidins, anhydroflavenols, catechins and chemical derivatives of these groups.

Research has suggested that isoflavones may inhibit the growth of human cancer cells. See, e.g., Setchell, K. D. R., and Welch, M. B., J. Chrom., 386 (1987), pp. 315-323; High Performance Liquid Chromatographic Analysis of Phytoestrogens in Soy Protein Preparations with Ultraviolet, Electrochemical and Thermospray Mass Spectrometric Detection, McLachlan, J. A., ed. Estrogens in the Environment, New York: Elsevier Press, 1985, pp. 69-85; and Setchell, et al., “Nonsteroidal Estrogens of Dietary Origin: Possible Roles in Hormone Dependent Disease,” Am. J. Clin. Nutr., 1984, 40, pp. 569-578. There is also some indirect, demographic support for an isoflavone mediated reduction in cancers of hormone responsive tissues based on observations that women in countries consuming vegetarian diets have a lower incidence of breast cancer compared to women in meat-eating countries. Adlercreutz et al., “Determination of Urinary Lignans and Phytoestrogen Metabolites, Potential Antiestrogens and Anticarcinogens, in Urine of Women on Various Habitual Diets,” Steroid. Biochem., 1986, 25, pp. 791-797. Isoflavones have also been suggested to have antiviral and fungicidal properties. And, they have been implicated in the reduction of serum cholesterol in humans, positive immunological effects and activity as an antioxidant. Isoflavones may also be useful as an alleviator of vasomotor symptoms in menopausal women, and have been used historically in Chinese medicine to treat “hot flashes.”

Plant proteins also contain significant amounts of phytates, accounting for as much as 85% of the total phosphorus in certain plants. One phytate is phytic acid. Phytic acid is also known as inositol hexaphosphate. As used herein, the term “phytates” means phytic acid and its isomers, the salts and derivatives of phytic acid and its isomers, and/or partially dephosphorylated isomers of phytic acid, and salts and derivatives of partially dephosphorylated isomers of phytic acid. Phytic acid serves several physiological functions and influences the functional and nutritional properties of cereals and vegetables by its ability to complex with both proteins and essential minerals. See, e.g., Ceryan, M., CRC Crit. Rev. Food Sci., Nutr., vol. 13, 297, 1980 and Graf, E., J. Am. Oil Chem. Soc., vol. 60, 1861, 1983. Phytic acid is also reported to be effective in preventing cancer. See, e.g., Reddy, B. S. et al., Cancer Res., vol. 60, no. 17, 2000, pp. 4792-4797; Shamsuddin, A. M. and Vucenik, I., Anticancer Res., vol. 19, no. 5A, 1999, pp. 3671-3674. Such properties have prompted research into methods for removal of phytates from plant sources, such as soy proteins. U.S. Pat. No. 5,213,835 to Nardelli et al., discloses a process for removing phosphorus from milk and whey proteins.

Purification of plant proteins and/or isolation of compounds such as isoflavones and phytates may be achieved by use of such methods as ion exchange technology. Such methods are disclosed, for example, in U.S. Pat. Nos. 5,985,338, 5,804,234, and 600,020,471, which are herein incorporated-by-reference. In such methods, purification of the plant proteins or removal of phytoestrogens or plant estrogens, manganese or nucleotides is effected by passing an aqueous slurry of an isoflavone containing material over an active surface such as an anion exchange resin, thereby binding such compound or compounds to the active surface. As those of skill in the art can appreciate, exchange resins and active surfaces have a finite capacity, but may be regenerated to an active state after exhaustion or near-exhaustion. After a certain amount of use, reconditioning of the active surface, or ion exchange resin will be useful. Such regeneration or reconditioning generally comprises removal of the bound compound or compounds from the active surface.

U.S. Pat. No. 5,804,234 to Suh et al., discloses a method for regenerating or reconditioning exchange resins (or removing the bound compounds) after contact with plant protein by contact with a salt solution comprising 6% NaOH, 1% HCl and 1.5% NaHCO3.

U.S. Pat. No. 6,020,471 to Johns et al., discloses a method for rinsing or releasing bound isoflavones from an ion exchange resin by contact with an aqueous alcohol solution.

U.S. Pat. No. 6,146,668 to Kelly et al., discloses a non-chromatographic approach to recovering isoflavones from plant material, and requires the use of organic solvents (e.g., ethyl acetate, hexane, acetone). The residual levels of such organic solvent would constitute a safety concern in the utilization of the recovered isoflavones in foods for human consumption. Kelly et al. makes no provision for phytate removal.

U.S. Pat. No. 6,171,638 to Gugger et al., discloses an ion exchange process for separation and purification of isoflavones, utilizing aqueous alcohol, but makes no provision for phytate removal.

U.S. Pat. No. 5,789,581 to Matsuura et al., discloses a process for obtaining certain isoflavones which comprises the use of an aqueous alcohol solution as an eluant.

U.S. Pat. No. 5,670,632 to Chaihorsky, discloses a process for recovering isoflavones from a soy extract which comprises the use of a highly polar sulfonic acid cationic exchange resin as an adsorbent and the use of an acidic alkanol containing 1 to 3 carbon atoms to facilitate desorption of the isoflavone 7-glycosides. The solution utilized in desorption is prepared with a concentrated alcohol (i.e., 96%) to yield a solution of 86% aqueous alcohol. The patent makes no provision for the recovery of phytates.

U.S. Pat. No. 5,506,211 to Barnes et al., discloses the use of a particular isoflavone (genistein) as an inhibitor of osteoclasts. Barnes et al. describes the aqueous extraction of isoflavones using 80% aqueous methanol.

U.S. Pat. No. 4,428,876 to Iwamura discloses a process for isolating saponins and flavonoids from leguminous plants which comprises the use of a polar solvent (such as methanol or aqueous methanol) to elute the saponins and flavonoids adsorbed on a resin.

### SUMMARY

Isoflavones and phytates can be sequentially removed from an active surface by utilizing a method whereby the active surface(s) are contacted with an aqueous medium for isoflavone removal and separately contacted with an aqueous medium for phytate removal. The aqueous medium for isoflavone removal comprises an acidic aqueous alcohol solution. The aqueous medium for phytate removal comprises either a relatively stronger aqueous acid solution or an aqueous basic solution. Alternatively, for use with some active surfaces, the aqueous medium for phytate removal may comprise an aqueous solution with a pH between about 2 and about 7 which is essentially free of alcohol and organic solvents. By this method, isoflavones and phytates are separately and sequentially removed and may be recovered. The recovered isoflavones and phytates may be utilized in products for human consumption, such as nutritional products or cereals.

### DETAILED DESCRIPTION

Methods are disclosed for sequentially removing isoflavones and phytates from active surface(s) comprising contacting the active surface with an aqueous medium for isoflavone removal and separately with an aqueous medium for phytate removal. The aqueous medium for isoflavone removal comprises an acidic aqueous alcohol solution. The aqueous medium for phytate removal comprises either a relatively stronger (i.e., lower pH) aqueous acid solution or an aqueous basic solution, or with some active surfaces may comprise an aqueous solution with a pH between about 2 and about 7 which is essentially free of alcohol and organic solvents.

The acid utilized in the aqueous medium for isoflavone may be selected from various acids, including, but not limited to, acetic acid, citric acid, hydrochloric acid, sulfuric acid, nitric acid, phosphoric acid, succinic acid, malic acid, malonic acid, tartaric acid, lactic acid, pyruvic acid, fumaric acid and mixtures thereof. Table I provides a comparison of isoflavone recovery with various acids utilized in the aqueous medium for isoflavone removal. The alcohol(s) utilized in the aqueous medium for isoflavone removal may be selected from various alcohols, including, but not limited to, methanol, ethanol, propanol and butanol, and mixtures thereof. Various relative amounts of acid and alcohol will be appropriate for use in the aqueous medium for isoflavone removal, depending upon factors such as the strength of the acid and the intended end use of the compound isolated. The amount of alcohol present in the aqueous medium for isoflavone removal may be between about 10 and about 90% (v/v). More preferably, the amount of alcohol present is between about 50 and about 70% (v/v). Table II provides a comparison of isoflavone recovery utilizing citric acid in the aqueous medium for isoflavone removal along with varying concentrations of alcohol. The amount of acid present in the aqueous medium for isoflavone removal will vary according to the strength of the acid (pKa) and concentration of acid utilized in preparing the medium, but generally should be an amount sufficient for the pH of the aqueous medium to be between about 1.5 and about 3.5. The amount of acid required to bring the aqueous medium for isoflavone removal to within such a pH range can easily be calculated by one of ordinary skill in the art. Generally amounts between about 0.1 and about 40% (w/w) are sufficient. As an example, when glacial acetic acid is utilized in the aqueous medium for isoflavone removal, it is preferably present in an amount from about 5 to about 40% (v/v), and more preferably in an amount from about 20 to about 30% (v/v). Table III provides a comparison of isoflavone recovery utilizing an aqueous medium for isoflavone removal with varying amounts of ethanol and glacial acetic acid. Table IV provides a comparison of isoflavone recovery utilizing an aqueous medium for isoflavone removal with 60% ethanol and varying amounts of glacial acetic acid. When citric acid is utilized in the aqueous medium for isoflavone removal, it is preferably present in an amount from about 10 to about 40 grams/liter, and more preferably in an amount from about 20 to about 30 grams/liter. A combination of more than one acid may also be utilized in the aqueous medium for isoflavone removal. For example, a suitable aqueous medium for isoflavone removal could contain approximately 10% glacial acetic acid and approximately 10 g/liter citric acid, along with approximately 60% reagent alcohol (reagent alcohol is denatured ethanol; the terms reagent alcohol and ethanol are used interchangeably herein). Table V illustrates comparative recovery of isoflavones utilizing a combination of more than one acid in the aqueous medium for isoflavone removal.

In another embodiment, the aqueous medium for isoflavone removal comprises more than one aqueous solution. For example, a first aqueous solution and a second aqueous solution may be used. The first aqueous solution may comprise an acid aqueous solution (with a pH between about 2 and about 7) essentially free of alcohol, and the second aqueous solution may comprise an aqueous alcohol solution essentially free of added acid. The method of contacting comprises first contacting the active surfaces with the first aqueous solution, and subsequently contacting the active surfaces with the second aqueous solution. The first aqueous solution contains no alcohol or organic solvent, but may contain at least one acid selected from the group consisting of citric acid, hydrochloric acid, sulfuric acid, nitric acid, phosphoric acid, succinic acid, malic acid, malonic acid, tartaric acid, lactic acid, pyruvic acid, fumaric acid and mixtures thereof. The pH of the first aqueous solution ranges from about 2 to about 7. More preferably, the pH of the first aqueous solution ranges from about 3 to about 6. The second aqueous solution may comprise at least one alcohol selected from the group consisting of methanol, ethanol, propanol, butanol, and mixtures thereof, but is essentially free of added acid. This second aqueous solution preferably comprises about 10 to about 90% (v/v) alcohol, more preferably from about 50 to about 80% (v/v) alcohol.

Generally, the aqueous medium for phytate removal comprises either a relatively stronger aqueous acid solution or an aqueous base solution. Alternatively, as discussed below, when used with some active surfaces, the aqueous medium for phytate removal may comprise an aqueous solution with a pH of about 2 to about 7 which is essentially free of alcohol and organic solvents. The acid utilized in the aqueous medium for phytate removal may be selected from various acids, including, but not limited to acetic acid, citric acid, hydrochloric acid, sulfuric acid, nitric acid, phosphoric acid, succinic acid, malic acid, malonic acid, tartaric acid, lactic acid, pyruvic acid, fumaric acid, and mixtures thereof. Table VI provides a comparison of phytic acid removal with various acids utilized in the aqueous medium for phytate removal. When the aqueous medium for phytate removal comprises an aqueous base solution, the base may be selected from various bases including, but not limited to, sodium hydroxide, potassium hydroxide, ammonium hydroxide, lithium hydroxide, sodium carbonate, and mixtures thereof. For pH values in the acidic range (i.e., less than 7), the relative amount of phytates removed from an active surface increases with decreasing pH, see Table VII, which illustrates phytic acid and isoflavone recovery at various pH values (hydrochloric acid concentrations). The amount of acid or base utilized in the aqueous medium for phytate removal will vary according to the strength of the acid (pKa) or base (pKb), and concentration of acid or base utilized in preparing the medium, but generally should be an amount sufficient for the pH of the aqueous medium for phytate removal to be less than about 1, more preferably about 0.1 to about 1.0 for an acid solution or alternatively about 13 to about 14 for a basic solution. The amount of acid or base required to bring the aqueous medium for phytate removal to within such a pH range can easily be calculated by one of ordinary skill in the art. As an example, when hydrochloric acid is utilized in the aqueous medium for phytate removal, the amount present is preferably sufficient so that the medium is about 0.2 to about 2 M HCl, and more preferably about 0.4 to about 0.8 M HCl. When a base is utilized in the aqueous medium for phytate removal, it is preferably present in an amount from about 2 to about 10% (w/w), and more preferably in an amount from about 4 to about 6% (w/w).

The particular pH that is chosen for both the aqueous medium for isoflavone removal and the aqueous medium for phytate removal may be varied according to the use which the isolated isoflavones and phytates will be put. Additionally, the amount of time that the aqueous medium for isoflavone removal and the aqueous medium for phytate removal are in contact with the active surfaces and the amount of each medium that is utilized may be varied. Generally, the total amount of isoflavone or phytate recovered increases as the contact time with the active surfaces increases and as the amount of medium that is utilized increases. However, such increased recovery must be balanced with other factors, including the cost of the protein (isoflavone and phytate source), disposal or treatment costs for additional solvents generated when increased volumes of media are utilized, costs for concentrating the isoflavones or phytates, and life time and cost of the active surfaces. In the examples provided below and in the tables provided herein, various contact times and volumes of media are utilized. Volumes of media are expressed herein as bed volumes or column volumes with contact times expressed in terms of bed volumes or column volumes per hour or per minute.

The active surface from which the isoflavones and phytates are removed and isolated is preferably an anion exchange resin. Suitable anion exchange resins are macroporous resins, preferably a Type I or Type II macroporous resin. For anion exchange chromatography, the anion exchange resin is selected from weak base anion exchange resins, strong base anion exchange resins and mixtures thereof. Representative examples include Amberlite□ RA95, IRA-910 and IRA-900 (available from Rohn and Haas Company), Dowex-22 and MSA-1 (available from Dow Chemical), and Purolite A510 and A500 (available from Purolite Company). As used herein, the term resin is meant to include gels, which those skilled in the art would understand to be useful in the process described herein. Representative gels include Amberlite□ IRA 410 (Type II gel, strong base anion) (available from Rohm and Haas) and IRA 402 (Type II gel, strong base anion exchange, not macroporous). The anion exchange resin may be contained within a column which has at least one inlet and at least one outlet, with the inlet located lower in the column structure than the outlet. Such a setup allows for a slurry of plant protein to be passed through the column and over the resin by entering the column through the inlet and exiting through the outlet. After the desired amount of plant protein slurry has been passed through the column, the resin is then contacted with the aqueous medium for isoflavone removal, prepared in the manner described above and the eluate is collected. After a desired amount of isoflavone eluate has been collected, the aqueous medium for phytate removal, prepared in the manner described above, may be passed through the column and contacted with the resin. The second eluate which is collected will contain phytates. Table VIII provides a comparison of the amounts of isoflavones and phytic acid sequentially recovered utilizing various combinations of aqueous medium for isoflavone removal and aqueous medium for phytate removal.

The active surface may also comprise an alkylsilane bonded phase medium. Alkylsilane bonded phase media are chromatography column packings made by chemically bonding an alkylsilane (e.g., octadecylsilane [ODS or C18] or octylsilane [C8] or butylsilane [C4]) to the surface of silica. When such are utilized, they are preferably contained within a column which has at least one inlet and at least one outlet, with the inlet located higher in the structure than the outlet. A representative example is Sep-Pak C18, an octadecylsilane, 55-105 micrometer diameter, 125 A pore size, 12% carbon (available from Waters Corporation). When the active surface comprises an alkylsilane bonded phase medium, the aqueous medium for phytate removal comprises an aqueous solution with a pH between about 2 and about 7 which is essentially free of alcohol and organic solvents. As used herein, the term organic solvents includes solvents such as ethanol, methanol, propanol, isopropanol, acetone, dimethylsulfoxide (D)MSO), dimethylformamide (DMIE), tetrahydrofuran CHF), dichloromethane, and etc. Suitable solutions for phytate removal for use with an alkylsilane phase bonded medium include, but are not limited to, water and various buffers with an appropriate pH. The aqueous medium for phytate removal may comprise a sufficient amount of an acid selected from the group consisting of acetic acid, citric acid, hydrochloric acid, sulfuric acid, nitric acid, phosphoric acid, succinic acid, malic acid, malonic acid, tartaric acid, lactic acid, pyruvic acid, fumaric acid and mixtures thereof in an amount sufficient to the solution to have a pH between about 2 and about 7, and is essentially free of alcohol and organic solvents. The aqueous medium for isoflavone removal for use with an alkylsilane bonded phase medium comprises an acidic aqueous alcohol solution, as generally described above. The acid utilized in the aqueous medium for isoflavone may be selected from various acids, including, but not limited to, acetic acid, citric acid, hydrochloric acid, sulfuric acid, nitric acid, phosphoric acid, succinic acid, malic acid, malonic acid, tartaric acid, lactic acid, pyruvic acid, fumaric acid and mixtures thereof. The alcohol(s) utilized in the aqueous medium for isoflavone removal may be selected from various alcohols, including, but not limited to, methanol, ethanol, propanol and butanol, and mixtures thereof. Various relative amounts of acid and alcohol will be appropriate for use in the aqueous medium for isoflavone removal, depending upon factors such as the strength of the acid and the intended end use of the compound isolated. The amount of alcohol present in the aqueous medium for isoflavone removal may be between about 10 and about 90% (v/v). More preferably, when utilized with an alkylsilane bonded phase medium, the amount of alcohol present is between about 50 and about 80% (v/v). As discussed above, the amount of acid present in the aqueous medium for isoflavone removal will vary according to the strength of the acid (pKa) and concentration of acid utilized in preparing the medium, but when utilized with an alkylsilane bonded phase medium generally should be an amount sufficient for the pH of the aqueous medium to be between about 2 and about 7. The amount of acid required to bring the aqueous medium for isoflavone removal to within such a pH range can easily be calculated by one of ordinary skill in the art. Generally amounts between about 0.1 and about 40% (w/w) are sufficient. As an example, when glacial acetic acid is utilized in the aqueous medium for isoflavone removal, it is preferably present in an amount from about 5 to about 40% (v/v), and more preferably in an amount from about 20 to about 30% (v/v). When citric acid is utilized in the aqueous medium for isoflavone removal, it is preferably present in an amount from about 10 to about 40 grams/liter, and more preferably in an amount from about 20 to about 30 grams/liter. A combination of more than one acid may also be utilized in the aqueous medium for isoflavone removal. Additionally, when the active surface comprises an alkylsilane bonded phase medium, the medium will be contacted first with the aqueous medium for phytate removal, and then separately contacted with an aqueous medium for isoflavone removal.

Additionally and alternatively, the active surfaces may comprise both anion exchange resin and alkylsilane bonded phase medium. When such are utilized, they are preferably contained within a column which has at least one inlet and at least one outlet, with the inlet located lower in the structure than the outlet. Preferably, the alkylsilane bonded phase medium is positioned closer to the inlet than said anion exchange resin, so that when an aqueous medium is passed through the column it contacts the alkylsilane bonded phase medium before it contacts the anion exchange resin. Aqueous media for isoflavone removal and for phytate removal, as described generally above may be utilized. More specifically, the aqueous medium for isoflavone removal comprises an acidic aqueous alcohol solution and the aqueous medium for phytate removal comprises either a relatively stronger (i.e., lower pH) aqueous acid solution or an aqueous basic solution. In an application where both anion exchange resin and alkylsilane bonded phase medium are utilized as the active surfaces, the pH of the aqueous medium for isoflavone removal is preferably between about 2 and 3.5.

Various mixtures of plant protein may be utilized for isolation of isoflavones and phytates. Commercially available mixtures may be utilized or custom blended mixtures may be prepared. One commercially available plant protein mixture is a soy protein isolate known as Ardex F□ (available from Archer Daniels Midland, Inc.). Other materials that may be used to supply the source of isoflavones and phytates include any material that contains a detectable level of isoflavones and phytates. Such materials include protein obtainable from soybeans, corn, wheat, peas, beans, cottonseed, peanuts, carrots, alfalfa, algae, potatoes, apples, barley, bluegrass, clovers, coffee, garlic, hops, marijuana, oats, orchard grass, parsley, rice, rye, sage, sesame, yeast, fungus, hydrolyzates thereof, and mixtures thereof.

Methods described herein may be conducted at room temperature, or may be conducted at elevated temperatures, such as at about 90° to about 120° F. For purposes of this invention, room temperature is defined as between about 60° F. and about 80° F. The methods may also be conducted at other temperatures. There is no preference on temperature, although as one of ordinary skill in the art will recognize, lower and upper limits will necessarily be restrained by the freezing points and boiling points of the various solutions utilized.

Processes whereby active surfaces, such as an anion exchange resin or an alkylsilane bonded phase medium, are utilized to purify plant proteins will be readily recognizable to those of ordinary skill in the art. Examples of such processes and details regarding such processes are provided in U.S. Pat. Nos. 5,985,338, 5,804,234, and 600,020,471. The methods claimed in this patent are not intended to be limited to methods for sequentially removing isoflavones and phytic acid from surfaces disclosed or described in those patents.

### EXAMPLES

Isoflavones and phytates can be sequentially isolated by methods within the scope of the claims by the following procedures. These examples are being presented as illustrations and should not be interpreted as limiting in any way.

### Example A

Method Utilizing Various Eluants

IEX Resin Preparation: 22 grams of IRA-910 anion exchange resin were weighed into a 250 milliliter beaker. Using laboratory water (i.e., purified water (distilled, deionized, demineralized), the resin was transferred into a 2.5×10 cm glass open column (Bio-Rad catalog no. 737-2511) equipped with a 2-way stopcock (Bio-Rad catalog no. 732-8102). (The terms laboratory water, purified water and deionized water are used interchangeably herein.) The following solutions were then passed through the resin: 45 milliliters of 6% NaOH in approximately 20 minutes; followed by 90 milliliters of laboratory water in approximately 20 minutes; followed by 100 milliliters of 1% HCl (0.12 M HCl) in approximately 30 minutes; 180 milliliters of laboratory water in approximately 40 minutes; 55 milliliters of 1.4% NaHCO₃ in approximately 30 minutes; and 360 milliliters of laboratory water in approximately 80 minutes.

IEX Column Preparation: 22.0 grams of Commodity 1922 (PTI soy protein isolate) were thoroughly suspended in 250 milliliters of laboratory water in a 400 milliliter beaker. A 1.0-1.5 gram sample was removed for isoflavone determination. 22 grams of prepared resin (from procedure above) were added to the soy protein isolate suspension and stirred gently for 180 minutes. The resin was allowed to settle to the bottom of the beaker, and another 2.00 milliliter aliquot sample was removed for isoflavone determination. The soy protein isolate suspension was decanted and discarded. The resin was rinsed with 4×250 milliliters of laboratory water, with decanting and discarding after each addition. Laboratory water was used to pour equal amounts of resin (approximately 7 grams) into each of three 2.5×10 cm glass open columns equipped with a 2-way stopcock. Each column was rinsed with 20 milliliters of laboratory water.

Three eluants were utilized, one for each column, as listed in Table I. The first eluant was 0.48 M HCl in 60% ethanol. The second eluant was 20% glacial acetic acid in 60% ethanol. The third eluant was 20 g/L citric acid in 60% ethanol. Each eluant was passed through one of the columns at a rate of 3.6 bed volumes per hour. A total of 14 bed volumes of eluant were passed through each column. Two fractions were collected, each consisting of the eluant from 7 bed volumes. The amounts of isoflavones recovered are listed in Table I.

Isoflavones were determined using gradient elution reverse phase HPLC. 5.00 milliliters of each fraction were diluted to 25 milliliters with laboratory water, and tested for isoflavones by the HPLC system described below:

### HPLC SYSTEM FOR ISOFTAVONE DETERMINATION

HPLC Column : Waters Nova-Pak C18, 3.9×150 mm, 4 um, 60A, Waters #86344

Mobile Phase A: 920 mL 0.02M KH₂PO₄, 80 mL acetonitrile; pH 3.1 with H₃PO₄

Mobile Phase B : 400 mL 0.02M KH₂PO₄, 600 mL acetonitrile; pH 3.1 with H₃PO₄

Flow Rate: 0.6 mL/minute

Column Temperature: 40° C.

Detection: UV at 262 nm and 250 nm

Injection: 20 uL

Run Time: 60 minutes

Elution Program:

| | | | | | | | |
|---|---|---|---|---|---|---|---|
| Time (minutes) | 0 | 5 | 40 | 42 | 45 | 48 | 60 |
| Mobile Phase B (%) | 0 | 0 | 42 | 100 | 100 | 0 | end |

### Example B

Method Utilizing Citric Acid and Various Amounts of Ethanol

IEX resin and columns were prepared as described above for Example A except that the soy protein isolate and resin suspension was stirred for 2 hours, and 5 columns were prepared, and each column contained 4.4 grams of resin. For this example, five eluants containing citric acid and varying amounts of ethanol were prepared and utilized. All eluants contained 20 g/L citric acid. The amounts of ethanol utilized in the five eluants were: 0% (v/v), 10% (v/v), 20% (v/v), 60% (v/v) and 90% (v/v).

As in Example A, each eluant was passed through one of the columns at a rate of 3.6 bed volumes per hour. A total of 14 bed volumes of eluant were passed through each column. Each eluate was tested for isoflavones according to the method described above in Example A. The amounts of isoflavones recovered are listed in Table II.

### Example C

Method Utilizing Various Amounts of Glacial Acetic Acid, Ethanol and Water

IEX Resin Preparation: The IEX Resin was prepared as described in Example A above.

IEX Column Preparation: 4.0 grams of soy protein isolate (PTI # C7H-XTO-9001) were thoroughly suspended in 200 milliliters of laboratory water in a 400 milliliter beaker. A 2.00 milliliter aliquot sample was removed for isoflavone determination (“column feed”). 20 grams of prepared resin (from procedure above) were added to the soy protein isolate suspension and stirred at approximately 400 rpm for 60 minutes. The resin was allowed to settle to the bottom of the beaker, and another 2.00 milliliter aliquot sample was removed for isoflavone determination (“unbound by IEX resin”). The soy protein isolate suspension was decanted and discarded. The resin was rinsed with 2×200 milliliters of laboratory water, with decanting and discarding after each addition. Laboratory water was used to resuspend the resin, and equal amounts of resin (approximately 3.3 grams) were poured into each of six 20 milliliters columns. Each column was rinsed with 10 milliliters of laboratory water. 15.0 milliliters of eluant was transferred by pipet into the column, and eluted at a flow rate of approximately 1 column volume per minute. A total of 5 column volumes of eluate were passed through each column. The eluate was tested for isoflavones according to the method described above in Example A.

Various eluants were prepared and utilized as listed in Table III. The eluants contained varying amounts of ethanol, water and glacial acetic acid. The amounts of isoflavones removed are listed in Table III.

### Example D

Method Utilizing Ethanol and Varying Amounts of Glacial Acetic Acid

IEX resin and columns were prepared as described above for Example B. For this example, five eluants containing ethanol and varying amounts of glacial acetic acid were prepared and utilized. All eluants contained 60% ethanol (v/v). The concentrations of glacial acetic acid present in the five eluants were: 0% (v/v), 10% (v/v), 20% (v/v), 30% (v/v), and 40% (v/v).

Each eluant was passed through one of the columns at a rate of approximately 10 column volumes per hour. A total of 10 bed volumes of eluant were passed through each column. The eluates were collected and each eluate was tested for isoflavones according to the method described above in Example A. The amounts of isoflavones recovered are listed in Table IV.

### Example E

Method Utilizing Various Eluants

IEX Resin and columns were prepared as described above for Example A.

For this example, three eluants were prepared and utilized. The first eluant contained 20 g/L citric acid in ⁶⁰% ethanol. The second contained 20% glacial acetic acid in 60% ethanol. The third contained 10 g/L citric acidtand 10% glacial acetic acid in 60% ethanol. Each eluant was passed through one of the columns at a rate of approximately 3.6 bed volumes per hour. A total of 14 bed volumes was passed through each column. The eluates were collected and tested for isoflavones. The amounts of isoflavones recovered are listed in Table V.

### Example F

Method Utilizing Various Eluants

IEX Resin and columns were prepared as described above in Example A, except that 6 columns were prepared, each column containing 3.6 grams of resin.

For this example, six eluants were prepared and utilized. The first contained 20% glacial acetic acid in 60% ethanol. The second contained 20% glacial acetic acid in water. The third contained 20 g/L citric acid in 60% ethanol. The fourth contained 20 g/L citric acid in water. The fifth contained 0.48 M HCl in 60% ethanol. The sixth contained 0.48 M HCl in water. Each eluant was passed through one of the columns at a rate of approximately 3.6 bed volumes per hour. A total of 14 bed volumes was passed through each column. The eluates were collected and each eluate was tested for phytic acid. The amounts of phytic acid recovered are listed in Table VI.

Phytic Acid was determined by the following procedure. For ethanolic eluate samples, 7.00 milliliters of the eluate was evaported to dryness with compressed N₂, and the residue resuspended in 7.00 milliliters of 0.02 M NaMalonate (pH 2.5). For aqueous eluate samples, the pH was adjusted to 2.5 with NaOH. 3.00 milliliters of eluate sample was transferred by pipet into each of two 1-dram vials. A phytase suspension was prepared by throughly suspending 36 mg of phyrate (Sigma catalog no. P-9792; crude, from Aspergillus ficum) in 4 milliliters of 0.02 M NaMalonate (pH 2.5). To the first vial was added 400 microliters of 0.02 M NaMalonate; this was the phorphorus control. To the second vial was added 400 microliters of the phytate suspension; this was the phytase digest. The vials were capped, mixed well, and incubated at 40° C. for 120 minutes. The vials were removed from the water bath. The sample suspensions were filtered through a 0.45 micrometer Acrodisc syringe filter (Gelman P/N 4497). The filtrate was tested for inorganic phosphorus, using Sigma's colorimetric Inorganic Phosphorus test kit (Sigma catalog no. 670-A) and a spectrophotometer. Eluate phytic acid was calculated by subtracting the non-phytic acid phosphorus (as measured in the phosphorus control) from the phytase digest phosphorus and by using the appropriate dilution factor and molecular weights for phosphorus (30.97) and for phytic acid (660.0).

### Example G

Method Utilizing Ethanol and Varying Amounts of HCl

IEX Resin Preparation: The IEX Resin was prepared as described in Example A above.

IEX Column Preparation: 22 grams of Commodity 1922 (PTI soy protein isolate “PP1610”) were thoroughly suspended in 250 milliliters of laboratory water in a 400 milliliter beaker. A 1.0-1.5 gram sample was removed for isoflavone determination. 22 grams of prepared resin (from procedure above) were added to the soy protein isolate suspension and stirred gently for 120 minutes. The resin was allowed to settle to the bottom of the beaker, and another 2.00 milliliters aliquot sample was removed for isoflavone determination. The soy protein isolate suspension was decanted and discarded. The resin was rinsed with 250 milliliters of laboratory water and decanted and the supernatant discarded. Rinsing was repeated until the resin was thoroughly rinsed of visible soy protein isolate solids. An equal quantity of resin (approximately 4.4 grams) was transferred into each of five 2.5×10 cm glass open columns (Bio-Rad # 737-2511) equipped with a 2-way stopcock (Bio-Rad #732-8102). Each column was rinsed with 50 milliliters of laboratory water. At this point, elution of isoflavones and/or phytates with desired eluants may begin.

For this example, five eluants were prepared and utilized. All contained 60 milliliters of ethanol and 40 milliliters of HCl in varying concentrations. HCl concentrations were 1.25 N, 1.00 N, 0.75 N, 0.50 N and 0.25 N. The entire volume of each eluant was passed through one of the columns at a rate of approximately 25 milliliters per hour. Each eluant was collected and analyzed for isoflavones, phytic acid and protein. The amounts of isoflavones, phytic acid and protein recovered are listed in Table VII.

Phytic acid was determined by pipetting 8.00 milliliters of column eluate into a 30 milliliter beaker. Ethanol was evaporated with a stream of compressed nitrogen. 15.0 milliliters of 0.05 M malonic acid were added to the beaker. The pH was adjusted to 2.5 with 2 N NaOH. The sample was diluted to 25 milliliters with 0.05 M malonic acid. 10.0 milliliters was pipetted into each of two vials. 5 mg of phytase (EC 3.1.3.8, Sigma # P-9792, 3.5 U/mg solid) was added to one of the two vials. Both vials were incubated at 37° C. for fourteen hours. The vials were cooled to room temperature and 10.0 milliliters of 20% trichloroacetic acid (w/v) was added to each vial. The vials were capped, mixed well and 3-4 milliliters were filtered through a 0.45 micrometer membrane (Gelman Acrodisc, P/N 4497). 1.00 milliliters of filtrate was tested for inorganic phosphorus by Sigma Test Kit 670-A (inorganic phosphorus; colonnetric endpoint method; Sigma 1999 Catalog).

Protein was determined by pipetting 5.00 milliliters of column eluate into a 2 dram vial. The sample was evaporated to dryness with a stream of compressed nitrogen. The residue was suspended in 2 milliliters of 6 M HCl and transferred into a 2 milliliter ampule. The ampule was nitrogen-blanketed, flame-sealed and heated at 110° C. for 22 hours. The ampule was cooled to room temperature and the sample evaporated to dryness. The residue was reconstituted in 2.00 milliliters of Beckman Na-S buffer and tested for amino acids on a Beckman Model 6300 Automated Amino Acid Analyzer (i.e., by ion exchange chromatography, post-column ninhydrin derivatization, and visible absorbance detection).

### Example H

Method Utilizing Sequential Eluants

IEX Resin and columns were prepared as described above in Example A.

For this example, three different sequential eluants were utilized, with varying amounts of each sequential eluant passed through the particular column. In the first column, three different eluants were utilized sequentially. The first eluant was 20% glacial acetic acid in 60% ethanol; 14 bed volumes of this eluant were utilized. The second eluant was 20% glacial acetic acid in water; 14 bed volumes of this eluant were utilized. The third eluant was 0.48 M HCl in water; two fractions of 20 bed volumes each were utilized.

In the second column, three different eluants were utilized sequentially. The first eluant was 20 g/L citric acid in 60% ethanol; 14 bed volumes of this eluant were utilized. The second eluant was 20 g/L citric acid in water; 14 bed volumes of this eluant were utilized. The third eluant was 0.48 M HCl in water; two fractions of 20 bed volumes each were utilized.

In the third column, two different eluants were utilized sequentially. The first eluant was 0.48 M HCl in 60% ethanol; 14 bed volumes of this eluant were utilized. The second eluant was 0.48 M HCl in water; two fractions were utilized, the first of 14 bed volumes and the second of 20 bed volumes.

All eluants were passed through the columns at approximately 3.6 bed volumes per hour. Eluates were collected and isoflavones and phytic acid measured by the procedures described above (Examples A and F). The amounts of isoflavones and phytic acid recovered are listed in Table VIII.

### Example I

Method Utilizing an Alkylsilane Active Surface with Various Eluants

Seven cartridges, each packed with 100 mg of octadecylsilane (C 18) bonded phase medium, were prepared by conditioning each with 20 bed volumes of methanol, and then rinsing each with 20 bed volumes of laboratory water. Onto each cartridge was loaded 1.00 milliliters of a 1% (w/w) slurry of soy protein isolate in laboratory water.

In this experiment, seven different eluants were prepared and utilized. The eluants were as follows: water; 20% glacial acetic acid in water; 20 g/L citric acid in water; ethanol; 20% glacial acetic acid in 60% ethanol; 20 g/L citric acid in 60% ethanol; and 10% glacial acetic acid and 10 g/L citric acid in 60% ethanol. Ten bed volumes of eluant were passed through each cartridge at a rate of four bed volumes per minute. Eluates were collected, and isoflavones measured by the procedure described above (Example A). The isoflavone recoveries are listed in Table IX.

### Example J

Method Utilizing Glacial Acetic Acid and Ethanol in the Aqueous Medium for Isofavone Removal and Hydrochloric Acid in the Aqueous Medium for Phytate Removal

From a well-stirred slurry of soy protein isolate (2.0 g of soy protein isolate in 100 milliliters of laboratory water) are removed 2 milliliters for control testing of the isoflavone and phytate contents. To the remainder of the suspension are added 10.0 g of anion exchange resin (IRA-910 in the chloride form). The whole is stirred magnetically for about 60 minutes, whereupon the suspension is decanted from the anion exchange resin beads. The latter are rapidly rinsed twice with laboratory water (100 milliliters) by decanting, and then slurried with a further 100 milliliters of laboratory water and transferred to a glass column chromatography tube (2.5×10 cm) equipped with a support frit and an outlet valve. The resin is rinsed with a further 100 milliliters of water, the eluant flow rate being adjusted to 0.5 column volumes/minute. Two hundred milliliters of an eluant (the aqueous medium for isoflavone removal) composed of ethanol, glacial acetic acid, and laboratory water in the ratio by volume of 3:1:1 are passed through the resin bed at 0.5 column volumes/minute. The column eluate is collected and tested for isoflavones by gradient elution reverse phase HPLC. Two hundred milliliters of an eluant (the aqueous medium for phytate removal) composed of 0.48M hydrochloric acid in deionized water are passed through the resin bed at 0.5 column volumes/minute. The column eluate is collected and tested for phytic acid by an enzymatic/colorimetric method (the enzyme phytase is used to selectively release phosphate from phytic acid, and the phosphate is then quantified as inorganic phosphorus by the phosphomolybdate colorimetric method).

### Example K

Method Utilizing Citric Acid and Ethanol in the Aqueous Medium for Isoflavone Removal and Sulfuric Acid in the Aqueous Medium for Phytate Removal

The procedure is followed as set forth in Example 1 except that the aqueous medium for isoflavone in Example J is replaced with 20 g/L citric acid in 60% (v/v) ethanol, and the aqueous medium for phytate removal is replaced with 0.24M sulfuric acid in deionized water.

### Example L

Method Utilizing Hydrochloric Acid and Ethanol in the Aqueous Medium for Isoflavone Removal and Sodium Hydroxide in the Aqueous Medium for Phytate Removal

The procedure is followed as set forth in Example J, except that the aqueous medium for isoflavone removal in Example J is replaced with 0.05M hydrochloric acid in 60% (v/v) ethanol, and the aqueous medium for isoflavone is replaced with 5% (w/v) sodium hydroxide in deionized water. The flow rate (for both aqueous media) is also changed to 2 column volumes/minute.

### Example M

Method Utilizing C18 Bonded Phase Medium

From a well-stirred slurry of soy protein isolate (2.0 g of soy protein isolate in 100 milliliters of laboratory water) is removed 2 milliliters for control testing of the isoflavone and phytate contents. A cartridge containing 100 mg of a C18 bonded phase medium is conditioned with 2 column volumes of ethanol and two column volumes of laboratory water. One milliliter of the soy protein isolate suspension is passed through the C 18 bonded phase medium at a flow rate of 1 column volume/minute. The column is rinsed with one milliliter of laboratory water. The column eluate (i.e., from the load+the rinse) is collected and tested for phytic acid by an enzymatic/colorimetric method (as described above). Two milliliters of an eluant composed of 20 g/L of citric acid in ⁶⁰% ethanol are passed through the cartridge at a flow rate of 1 column volume/minute. The column eluate is collected and tested for isoflavones by gradient elution reverse phase HPLC.

Particular embodiments have been described above that fall within the scope of the invention as set forth in the claims. These embodiments are not intended to limit the scope of the invention to the specific forms disclosed. The invention is intended to cover all modifications and alternative forms falling within the spirit and scope of the invention.

TABLE I
Isoflavone Recovery Comparison

| | Isoflavone Recovery (% of IEX Bound) | | |
|---|---|---|---|
| | Fraction 1 | Fraction 2 | Total |
| IEX* Eluant Composition | (7 BV)** | (7 BV)** | (14 BV)** |
| 0.48 M HCl in 60% ethanol | 46 | 18 | 64 |
| 20% glacial acetic acid in 60% | 46 | 16 | 62 |
| ethanol | | | |
| 20 g/L citric acid in 60% ethanol | 36 | 26 | 62 |

*IEX = ion exchange resin
**BV = bed volumes of eluant; flow rate = 3.6 bed volumes per hour

TABLE II
Isoflavone Recovery vs. Eluant Alcohol Concentration -
Isoflavone Elution from IRA-910 IEX Column.

| | Isoflavone |
|---|---|
| | Recovery (% |
| IEX Column Eluant Composition** | of IEX Bound) |
| 20 g/L citric acid monohydrate in 0% ethanol (v/v) | 7 |
| 20 g/L citric acid monohydrate in 10% ethanol (v/v) | 12 |
| 20 g/L citric acid monohydrate in 20% ethanol (v/v) | 19 |
| 20 g/L citric acid monohydrate in 60% ethanol (v/v) | 64 |
| 20 g/L citric acid monohydrate in 90% ethanol (v/v) | 49 |

*all eluants were prepared with laboratory water and isoflavones were eluted with 14 bed volumes at a rate of 3.6 bed volumes per hour.

TABLE III
Isoflavone Removal vs. Eluant Composition

| | Isoflavones* Removed |
|---|---|
| | by 5 CV** |
| Eluant Composition | at 1 CV/minute (% of |
| (percentages are volume basis) | IEX Bound) |
| 50% ethanol, 20% glacial acetic acid, 30% water | 46.6 |
| 60% ethanol, 20% glacial acetic acid, 20% water | 49.4 |
| 70% ethanol, 20% glacial acetic acid, 10% water | 38.3 |
| 80% ethanol, 20% glacial acetic acid, 0% water | 18.4 |
| 60% ethanol, 30% glacial acetic acid, 10% water | 41.0 |
| 60% ethanol, 40% glacial acetic acid, 0% water | 39.7 |

*Isoflavones measured were daidzein and genistein.
**column volumes

TABLE IV
Isoflavone Recovery vs. Eluant Acetic Acid

| | Isoflavones | Isoflavone |
|---|---|---|
| Eluant Composition | in 10 CV* | Recovery** |
| 60% ethanol, 0% glacial acetic acid | 5.0 mg/L | 9.3% |
| 60% ethanol, 10% glacial acetic acid | 24.7 mg/L | 46.2% |
| 60% ethanol, 20% glacial acetic acid | 29.3 mg/L | 54.8% |
| 60% ethanol, 30% glacial acetic acid | 28.8 mg/L | 53.95% |
| 60% ethanol, 40% glacial acetic acid | 20.6 mg/L | 38.4% |

*10 column volumes of eluant at a rate of approximately 10 column volumes per hour.
**recovery of total aglycones bound by the IRA-910 IEX resin

TABLE V
Isoflavone Recovery from IRA-910 IEX Column

| | Isoflavone |
|---|---|
| | Recovery (% of |
| IEX Column Eluant Composition* | IEX Bound) |
| 20 g/L citric acid monohydrate in 60% ethanol | 55 |
| 20% glacial acetic acid in 60% ethanol | 56 |
| 10 g/L citric acid in 10% glacial acetic acid in | 53 |
| 60% ethanol | |

*isoflavones were eluted with 14 bed volumes at a rate of 3.6 bed volumes per hour.

TABLE VI
Phytic Acid Removal Comparison

| | mg of |
|---|---|
| | Phytic Acid removed from |
| IEX Eluant Composition | IEX Column* |
| 20% glacial acetic acid in 60% ethanol | <1 |
| 20% glacial acetic acid in water | <1 |
| 20 g/L citric acid in 60% ethanol | <1 |
| 20 g/L citric acid in water | <1 |
| 0.48 M HCl in 60% ethanol | 19 |
| 0.48 M HCl in water | 30 |

*mg phytic acid removed by 14 bed volumes of eluant at a rate of 3.6 bed volumes per hour.

TABLE VII
IEX Elution vs. Hydrochloric Acid Concentration

| IEX Eluant | HCl Conc. | pH* | Isoflavones | Phytic Acid | Protein |
|---|---|---|---|---|---|
| A | 0.5 N | 0.30 | 857 μg | 22.7 mg | 11.6 mg |
| B | 0.4 N | 0.40 | 903 μg | 15.8 mg | 11.9 mg |
| C | 0.3 N | 0.52 | 820 μg | 9.35 mg | 10.2 mg |
| D | 0.2 N | 0.70 | 904 μg | 5.61 mg | 10.9 mg |
| E | 0.1 N | 1.00 | 805 μg | 2.53 mg | 9.5 mg |

*pH values are calculated as the negative log the hydrogen ion concentration.
“isoflavones” = aglycone sum of daidzein, glycitein, and genistein compounds
“protein” = (dehydrated) amino acid total after acid hydrolysis

TABLE VIII
Sequential Elution of Isoflavones and Phytic Acid

| | | | Isoflavone | Phytic |
|---|---|---|---|---|
| IEX | | # of | Recovery (% | Acid |
| Column | Eluant Composition | BV* | of IEX Bound) | (mg) |
| A | 20% glacial acetic acid in | 14 | 62 | <1 |
| | 60% ethanol | | | |
| | 20% glacial acetic acid in | 14 | — | <1 |
| | water | | | |
| | 0.48 M HCl in water | 20 | — | 50 |
| | 0.48 M HCl in water | 20 | — | 24 |
| B | 20 g/L citric acid | 14 | 62 | <1 |
| | monohydrate in 60% ethanol | | | |
| | 20 g/L citric acid | 14 | — | <1 |
| | monohydrate in water | | | |
| | 0.48 M HCl in water | 20 | — | 47 |
| | 0.48 M HCl in water | 20 | — | 22 |
| C | 0.48 M HCl in 60% ethanol | 14 | 64 | 19 |
| | 0.48 M HCl in water | 14 | — | 30 |
| | 0.48 M HCl in water | 20 | — | 20 |

*BV = bed volumes of eluant at a rate of approximately 3.6 bed volumes per hour.

TABLE IX
Isoflavone Recovery from C18 Bonded
Phase Medium - Eluant Recovery
Comparison

| | Isoflavone |
|---|---|
| | Recovery (% of C18 |
| Eluant (10 bed volumes) | Bound) |
| Water | <1% |
| 20% glacial acetic acid in water | 30% |
| 20 g/L citric acid in water | <1% |
| ethanol | 96% |
| 20% glacial acetic acid in 60% ethanol | 89% |
| 20 g/L citric acid in 60% ethanol | 92% |
| 10% glacial acetic acid, 10 g/L citric acid in 60% | 95% |
| ethanol | |

1. A method for the sequential removal of isoflavones and phytates from one, or more, active surfaces, comprising the steps of:
(a) providing active surfaces comprising an anion exchange resin;
(b) contacting said active surfaces with an aqueous medium for isoflavone removal; and
(c) contacting said active surfaces with an aqueous medium for phytate removal.

2. A method as defined in claim 1, wherein said active surfaces are contained within a column which has at least one inlet and at least one outlet, said inlet located lower in the column than said outlet, such that said aqueous medium for isoflavone may be contacted with said active surfaces and a first eluate collected, and then said aqueous medium for phytate removal may be contacted with said active surfaces and a second eluate collected.

3. A method as defined in claim 2, wherein said first eluate contains less than 1% (w/v) of phytate.

4. A method as defined in claim 2, wherein said method is conducted at room temperature.

5. A method as defined in claim 2, wherein said method is conducted at a temperature from 90° to 120° F.

6. A method as defined in claim 2, wherein said aqueous medium for isoflavone removal comprises:
(a) 1-40% (w/w) of at least one acid selected from the group consisting of acetic acid, citric acid, malic acid, malonic acid, lactic acid, and mixtures thereof; and
(b) 10-90% (v/v) of at least one alcohol selected from the group consisting of methanol, ethanol, propanol, butanol, and mixtures thereof.

7. A method as defined in claim 2, wherein said aqueous medium for isoflavone removal comprises 50-70% v/v of at least one alcohol selected from the group consisting of methanol, ethanol, propanol, butanol, and mixtures thereof.

8. A method as defined in claim 2, wherein said aqueous medium for isoflavone removal comprises:
(a) at least one acid selected from the group consisting of acetic acid, citric acid, hydrochloric acid, sulfuric acid, nitric acid, phosphoric acid, succinic acid, malic acid, malonic acid, tartaric acid, lactic acid, pyruvic acid, fumaric acid and mixtures thereof;
(b) 10-90% (v/v) of at least one alcohol selected from the group consisting of methanol, ethanol, propanol, butanol, and mixtures thereof;
wherein the aqueous medium has a pH ranging from 1.5 to 3.5.

9. A method as defined in claim 2, wherein said aqueous medium for phytate removal has a pH less than 1.

10. A method as defined in claim 2, wherein said aqueous medium for phytate removal comprises at least one acid selected from the group consisting of acetic acid, citric acid, hydrochloric acid, sulfuric acid, nitric acid, phosphoric acid, succinic acid, malic acid, malonic acid, tartaric acid, lactic acid, pyruvic acid, fumaric acid, and mixtures thereof, wherein the aqueous medium has a pH less than 1.

11. A method as defined in claim 2, wherein said aqueous medium for phytate removal has a pH between 13 and 14.

12. A method as defined in claim 2, wherein said aqueous medium for isoflavone removal comprises at least two separate aqueous solutions.

13. A method for the sequential removal of isoflavones and phytates active surfaces, comprising the steps of:
(a) providing active surfaces comprising alkylsilane bonded phase media;
(b) contacting said active surfaces with an aqueous medium for isoflavone removal; and
(c) contacting said active surfaces with an aqueous medium for phytate removal.

14. A method as defined in claim 13, wherein said alkylsilane bonded phase medium is contacted with an aqueous medium for phytate removal which comprises an aqueous solution with a pH of 2 to 7, essentially free of alcohol and organic solvents, and said alkylsilane bonded phase medium is then contacted with an aqueous medium for isoflavone removal.

15. (canceled).

16. (canceled)

17. A method for sequentially isolating isoflavones and phytates from plant materials comprising:
(a) providing at least one anion exchange resin;
(b) providing a slurry of plant protein that contains isoflavones and phytic acid;
(c) placing the anion exchange resin in a structure which has at least one inlet and at least one outlet, and said inlet is located lower in the structure than the outlet;
(d) passing the slurry into the structure and over the resin by entering through the inlet and exiting through the outlet;
(e) contacting said resin with an aqueous medium for isoflavone removal and collecting a first eluate; and
(f) then contacting the resin with an aqueous medium for phytate removal and collecting a second eluate.
